(19) United States
(12) Patent Application Publication
(10) Pub. No.: US 2018/0129763 A1
(43) Pub. Date: May 10, 2018

(54) METHOD OF MANUFACTURING FOOT AUXILIARY EQUPIMENT

(71) Applicant: INDUSTRIAL TECHNOLOGY RESEARCH INSTITUTE, Chutung (CN)

(72) Inventors: Ming-Kan LIANG, Hsinchu City (TW); Wei LI, Hsinchu City (TW); Chih-Ming SHEN, New Taipei City (TW); Ming-Ji DAI, Hsinchu City (TW); Chia-Wei JUI, Zhubei City (TW)

(21) Appl. No.: 15/373,761

(22) Filed: Dec. 9, 2016

(30) Foreign Application Priority Data
Nov. 10, 2016 (TW) 105136647

(51) Int. Cl. G06F 17/50; A43D 1/02; A61F 5/01; B33Y 80/00; B33Y 10/00; B33Y 50/02; B29C 67/00

(52) U.S. Cl. CPC G06F 17/5018; A43D 1/025; A61F 5/0195; B29L 2031/753; B33Y 10/00; B33Y 50/02; B29C 67/0088; B33Y 80/00

(57) ABSTRACT

A method of manufacturing a foot auxiliary equipment includes the following steps. Firstly, a foot appearance of a foot and a foot muscle of the foot are scanned for obtaining a foot appearance data model and a foot muscle data s model respectively. Then, the foot appearance data model and the foot muscle data model are synthesized into a foot data model. Then, a dynamic state analysis and a static state analysis are performed on the foot data model. Then, a foot auxiliary equipment data model is generated according to result of the dynamic state analysis and result of the static state analysis. Then, a foot auxiliary equipment is printed by using three-dimensional printing technique according to the foot auxiliary equipment data model.

### CROSS-REFERENCE TO RELATED APPLICATION

This application claims the benefit of Taiwan application Serial No. 105136647, filed Nov. 10, 2016, the subject matter of which is incorporated herein by reference.

### TECHNICAL FIELD

The technical field relates to a method of manufacturing a foot auxiliary equipment, and more particularly to method of manufacturing a foot auxiliary equipment by using a three-dimensional printing technology.

### BACKGROUND

In order to help patients whose foot is hurt, the foot auxiliary equipment is needed. In a conventional practice, a foot mold is made of gypsum having a mold cavity which defines the shape of the foot. Then, a false foot produced by using the foot casting, and then the auxiliary equipment using the false foot.

However, the problem with this approach is that it is often difficult to improve when the foot auxiliary equipment is finally found to be problematic. In addition, the foot auxiliary equipment produced by this prior art method can only contain a single material, which limits the design flexibility of the foot auxiliary equipment.

Thus, it is needed to provide a new technique to resolve above problem.

### SUMMARY OF THE DISCLOSURE

The present disclosure provides a method of manufacturing a foot auxiliary equipment capable of resolving the above problem.

According to an embodiment of the disclosure, a method of manufacturing foot auxiliary equipment is provided. The method includes the following steps. A method of manufacturing a foot auxiliary equipment includes the following steps. A foot appearance of a foot and a foot muscles of the foot are scanned for obtaining a foot appearance data model and a foot muscle data model respectively. The foot appearance data model, a foot bone data model and the foot muscle data model are synthesized into a foot data model. A dynamic state analysis and a static state analysis are performed on the foot data model. A foot auxiliary equipment data model is generated according to result of the dynamic state analysis and result of the static state analysis. A foot auxiliary equipment is printed by using three-dimensional printing technique according to the foot auxiliary equipment data model.

The above and other aspects of the present disclosure will become better understood with regard to the following detailed description of the preferred but non-limiting embodiment(s). The following description is made with reference to the accompanying drawings.

### BRIEF DESCRIPTION OF THE DRAWINGS

FIG. 1 illustrates a flowchart of manufacturing a foot auxiliary equipment according to an embodiment of the present disclosure;

FIG. 2 illustrates processes of a foot data model 20 according to an embodiment of the present disclosure;

FIG. 3 illustrates a diagram of a foot auxiliary equipment data model 30 lo according to an embodiment of the present disclosure;

FIG. 4 illustrates a diagram of a lightened foot auxiliary equipment data model 30′ of FIG. 3;

FIG. 5 illustrates a diagram of a surface-finished foot auxiliary equipment data model 30″ of FIG. 3;

FIG. 6 illustrates a diagram of filling in the foot auxiliary equipment data model 30′″ of FIG. 3 with several materials;

FIG. 7 illustrates a diagram of a wearing data model 40 according to an embodiment of the present disclosure; and

FIG. 8 illustrates a diagram of a foot auxiliary equipment 50 according to an embodiment of the present disclosure.

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

### DETAILED DESCRIPTION OF DISCLOSED EMBODIMENTS

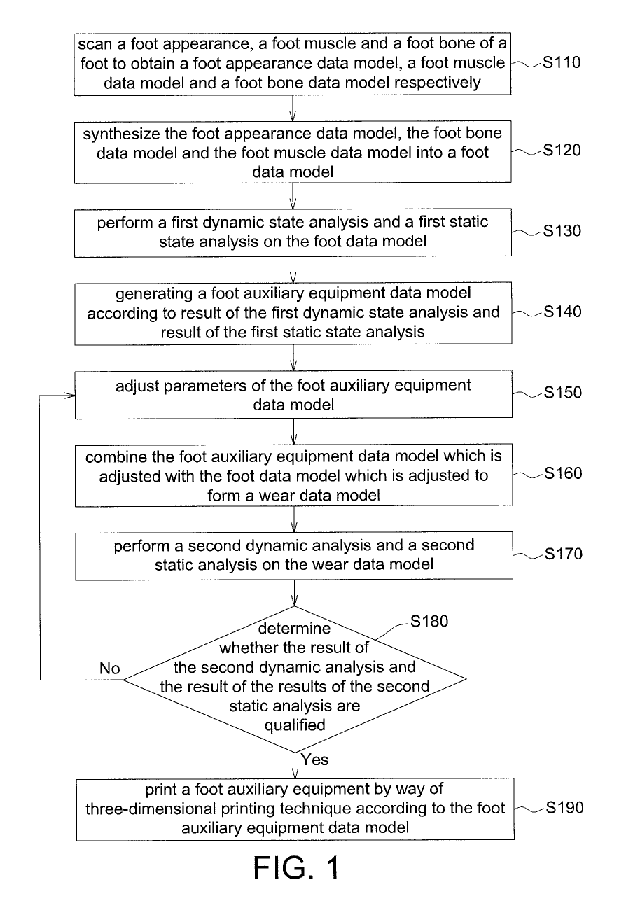

FIG. 1 illustrates a flowchart of manufacturing a foot auxiliary equipment according to an embodiment of the present disclosure.

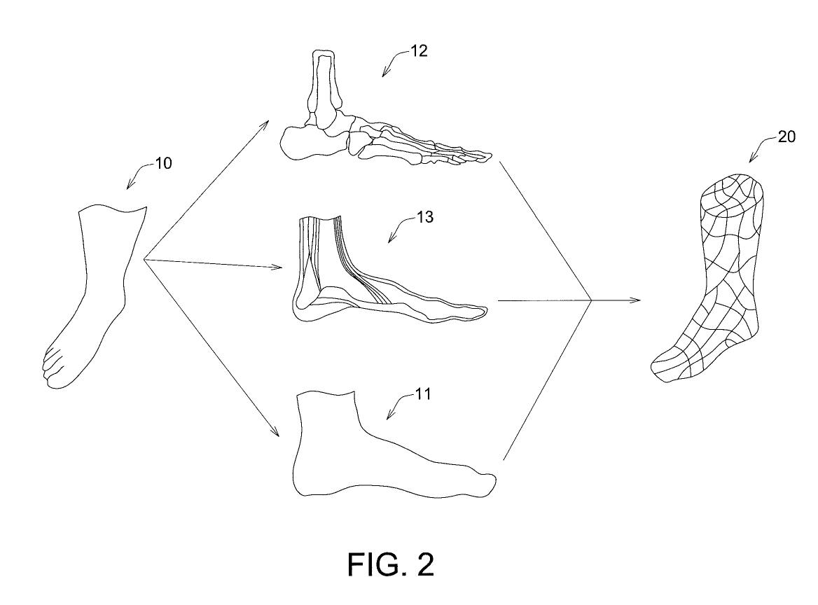

In step S110, referring to FIG. 2, FIG. 2 illustrates processes of a foot data model 20 according to an embodiment of the present disclosure. A foot appearance, a foot bone and a foot muscle of a foot 10 are scanned by using a three-dimensional (3D) image scanner to obtain a foot appearance data model 11, a foot bone data model 12 and a foot muscle data model 13 respectively. The 3D image scanner may be an appearance camera and an X-ray camera, wherein the appearance camera may capture the image of the foot appearance data model 11, and the X-ray camera may capture the image of the foot bone data model 12 and the image of the foot muscle data model 13. However, the 3D image scanner is not limited to the present embodiment of this disclosure. As long as a device can scan the foot appearance, the foot bone and the foot s muscle of the foot 10, it may serve as the 3D image scanner of the present disclosure. In addition, the foot appearance data model 11, the foot bone data model 12 and the foot muscle data model 13 may contain weight information for the purpose of the analysis. The weight information may be inputted manually or by calculated by a processor according to the volume of the foot appearance data model 11, the foot bone data model 12 and the foot muscle data model 13.

In another embodiment, the step of scanning the foot bone data model 12 may also be omitted.

In an embodiment, the foot appearance data model 11, the foot bone data model 12 and the foot muscle data model 13 may be displayed on a display screen (not illustrated) for making an operator to conveniently observe the foot appearance data model 11, the foot bone data model 12 and the foot muscle data model 13. Any model generated in the subsequent steps may be displayed on the display screen.

The foot appearance data model 11, the foot bone data model 12 and the foot muscle data model 13 each including a Computer Aided Design (CAD) model and a Finite Element Method (FEM) model. The CAD model can be used for subsequent manufacturing of a physical product, while the FEM model can be used for subsequent static analysis and dynamic analysis.

In step S120, as illustrated in FIG. 2, a processor (not illustrated) may synthesize the foot appearance data model 11, the foot bone data model 12 and the foot muscle data model 13 into the foot data model 20. The processor herein is, for example, a computer, a built-in Central Processing Unit (CPU) of a server or other relevant circuit manufactured by semiconductor manufacturing processes.

In step S130, the processor performs a first dynamic state analysis and a first static state analysis on the foot data model 20. The first static state analysis is, for example, a static analysis. For example, the exerted force situation of each portion of the foot data model 20 being at rest can be analyzed when the foot data model 20 is simulated to lie down or stand up. The first dynamic state analysis is, for example, gait analysis. For example, the exerted force situation of each portion of the foot data model 20 can be analyzed when the foot data model 20 is simulated to walk or run.

In another embodiment, the first dynamic state analysis and the first static state analysis can be performed on one of the foot appearance data model 11, the foot bone data model 12 and the foot muscle data model 13 for obtaining the individual CAD model and the individual FEM model.

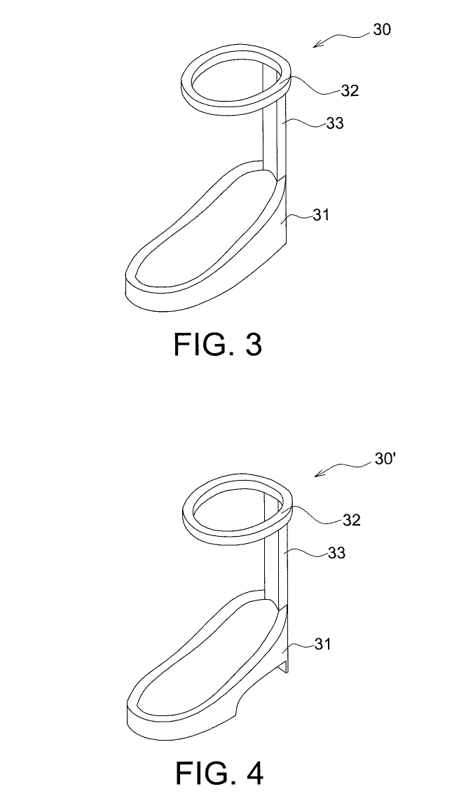

In step S140, as illustrated in FIG. 3, FIG. 3 illustrates a diagram of a foot auxiliary equipment data model 30 according to an embodiment of the present disclosure. The processor may generate the foot auxiliary equipment data model 30 according to result of the first dynamic state analysis and result of the first static state analysis. The foot auxiliary equipment data model 30 includes a foot pad portion 31, a support portion 32, and a connection portion 33, wherein the connection portion 33 connects the foot pad portion 31 and the support portion 32. In order to fit in with the appearance of the foot 10, the processor may design the support portion 32 to be shaped into a loop so that the foot 10 may pass through the support portion 32 and stabilize the wearing stability.

In step S150, the processor may adjust parameters of the foot auxiliary equipment data model 30. The parameters are, for example, weight, surface roughness, material or other parameters that may enhance wearing comfort and/or quality of remedy.

In an adjustment method, as illustrated in FIG. 4, FIG. 4 illustrates a diagram of a lightened foot auxiliary equipment data model 30′ of FIG. 3. The processor may reduce the weight of the foot auxiliary equipment data model 30 to obtain the lightweight foot auxiliary data model 30′. For example, the local thickness of the foot auxiliary equipment data model 30 may be thinned, for example, the foot pad portion 31. In another embodiment, the lightweight portion is not limited to the foot pad portion 31, and it also can be other portion of the foot auxiliary equipment data model 30. In addition, a sharp or a corner of the foot auxiliary equipment data model 30 may be rounded to reduce the weight of the foot auxiliary equipment data model 30 and avoid the discomfort in wearing caused by the sharp or the corner.

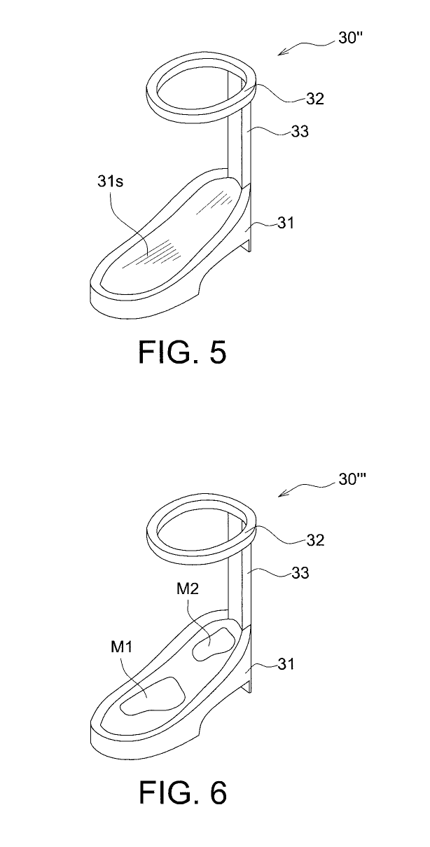

In another embodiment, as illustrated in FIG. 5, FIG. 5 illustrates a diagram of a surface-finished foot auxiliary equipment data model 30″ of FIG. 3. The processor may perform the surface treatment on the foot auxiliary equipment data model 30 for obtaining the surface-finished foot auxiliary equipment data to model 30″. For example, a surface 31s of the foot pad portion 31 which touches the bottom of the foot 10 may be smoothed, such that the manufactured foot auxiliary equipment provides a comfort in wearing.

In other embodiment, as illustrated in FIG. 6, FIG. 6 illustrates a diagram of filling in the foot auxiliary equipment data model 30″ of FIG. 3 with several is materials. The processor may fill in the foot auxiliary equipment data model 30 with different materials to obtain the foot auxiliary equipment data model 30′″ which is defined by the materials. For example, a first material M1 may be filled in a front portion of the foot pad portion 31 of the foot auxiliary equipment data model 30, and a second material M2 may be filled in a rear portion of the foot pad portion 31 of the foot auxiliary equipment data model 30, wherein the front portion and the rear portion bear larger force than other portion of the foot auxiliary equipment data model 30 does. The first material M1 and the second material M2 are, for example, Polyvinyl chloride (PVC), a viscoelastic material or other material suitable for the wearing of the foot 10.

The processor may determine the first material M1 and the second material M2 according the result of the first dynamic state analysis and the result of the first static state analysis. If the front portion of the foot pad portion 31 bears smaller force, the second material M2 may be made of softer material. If the rear portion of the foot pad portion 31 bears heavier force, the second material M2 may be made of harder material. In addition, the processor may fill in other portion of the foot auxiliary equipment data model 30 rather than the foot pad portion 31 with the rubber. In addition, the support portion 32 and the connection portion 33 may be may be filled in with a material including metal, polymer, etc.

Although the number of the adjustment methods as aforementioned embodiments is three, such exemplification is not meant to be for limiting. The aforementioned adjustment method is an optimization process. The purpose of the optimization process is for making the foot auxiliary equipment data model 30 to be the least weight and/or best fit for the human body based on the foot auxiliary equipment data model 30 with sufficient wear strength; however, such exemplification is not meant to be for limiting.

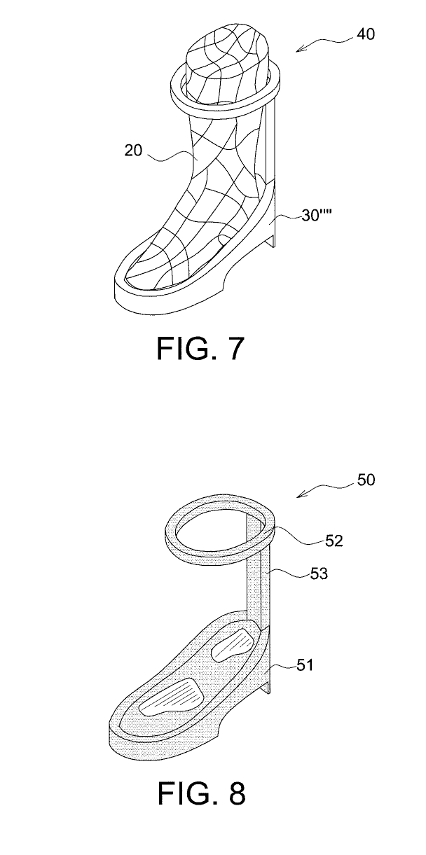

In step S160, as illustrated in FIG. 7, FIG. 7 illustrates a diagram of a wearing data model 40 according to an embodiment of the present disclosure. The processor may combine the foot auxiliary equipment data model 30′″ which is adjusted with the foot data model 20 which is adjusted to obtain the wearing s data model 40. In the present embodiment, the foot auxiliary equipment data model 30′″ of the present embodiment of the present disclosure is composed of the foot auxiliary equipment data model 30′ of FIG. 4, the foot auxiliary equipment data model 30″ of FIG. 5 and the foot auxiliary equipment data model 30′″ of FIG. 6.

In step S170, the processor performs a second dynamic state analysis and a second static state analysis. Since the physical foot auxiliary equipment has not produced yet, even if the result of the second dynamic state analysis and the result of the second static state analysis are disqualified, the process still can proceed to step S150 to make the processor to perform analysis again until the result of the second dynamic state analysis and the result of the second static state analysis are qualified. As a result, the cost of manufacturing and modifying the physical foot auxiliary equipment may be reduced or avoided.

In step S180, the processor determines whether the result of the second dynamic state analysis and the result of the second static state analysis are qualified. If the result of the second dynamic state analysis and the result of the second static state analysis are qualified, the process proceeds to step S190. If the result of the second dynamic state analysis and the result of the second static state analysis are not qualified, the process proceeds to step S150 to re-adjust or slightly adjust the parameters of the foot auxiliary equipment data model 30.

In step S190, as illustrated in FIG. 8, FIG. 8 illustrates a diagram of a foot auxiliary equipment 50 according to an embodiment of the present disclosure. If the result of the second dynamic state analysis and the result of the second static state analysis are qualified, the foot auxiliary equipment 50 is printed by 3D print technique according to the foot auxiliary equipment data model 30′″.

As described above, before the physical foot auxiliary equipment 50 is printed, the processor performs the simulation and the analysis repeatedly on the foot auxiliary equipment data model 30 and the wearing data model 40. When the result of the simulation and the result of the analysis are qualified, the physical foot auxiliary equipment 50 is started to be printed. As a result, the number of modifying the foot auxiliary equipment 50 and the cost of manufacturing the foot auxiliary equipment 50 may be reduced. In addition, compared with manually manufacturing method in prior art, due to the processor of the present disclosure has fast operating speed, the required time of manufacturing the foot auxiliary equipment of the present embodiment may be reduced. Furthermore, since the method of manufacturing the foot auxiliary equipment of the present embodiment has the advantages of rapid design and high design elasticity, it is possible to manufacture the customized foot auxiliary equipment for different patient's feet.

As illustrated in FIG. 8, the foot auxiliary equipment 50 includes a foot pad portion 51, a support portion 52 and a connection portion 53, wherein the connection portion 53 connects the pad portion 51 with the support portion 52. The size, the weight and the surface roughness of the foot pad portion 51, the support portion 52 and the connection portion 53 are similar to that of aforementioned the foot pad portion 31, the support portion 32 and the connection portion 33 of the foot auxiliary equipment data model 30′. In addition, compared with the foot auxiliary equipment produced by the prior art, due to the embodiment of the present disclosure uses 3D print technique, the foot auxiliary equipment 50 may be printed using different materials to make the foot auxiliary equipment 50 to become the auxiliary equipment with composite materials.

As described above, since the foot auxiliary equipment of the embodiment of the present disclosure is produced by 3D print technique, the foot auxiliary equipment is the auxiliary equipment with composite materials. In an embodiment, before the physical foot auxiliary equipment is printed, the 3D scanning may be performed on the patient's foot to obtain at least one foot data model, and then the dynamic state analysis and the static state analysis may be performed on the at least one foot data model to generate at least one foot auxiliary equipment data model. In another embodiment, before the foot auxiliary equipment is printed, the parameters of the foot auxiliary equipment may be adjusted, and then the dynamic state analysis and the static state analysis may be performed on one foot auxiliary equipment data model or combined foot auxiliary equipment data models to optimize the one foot auxiliary equipment data model or the combined foot auxiliary equipment data models.

It will be apparent to those skilled in the art that various modifications and variations can be made to the structure of the disclosed embodiments without departing from the scope or spirit of the disclosure. In view of the foregoing, it is intended that the disclosure cover modifications and variations of the present disclosure provided they fall within the scope of the following claims and their equivalents.

What is claimed is:

1. A method of manufacturing a foot auxiliary equipment, comprising:
scanning a foot appearance of a foot and a foot muscle of the foot to obtain a foot appearance data model and a foot muscle data model respectively;
synthesizing the foot appearance data model, a foot bone data model and the foot muscle data model into a foot data model;
performing a first dynamic state analysis and a first static state analysis on the foot data model;
generating a foot auxiliary equipment data model according to results of the first dynamic state analysis and the first static state analysis; and
printing a foot auxiliary equipment by three-dimensional (3D) printing technique according to the foot auxiliary equipment data model.

2. The method according to claim 1, wherein the step of scanning the foot appearance of the foot and the foot muscle of the foot further comprises:
scanning a foot bone of the foot to obtain a foot bone data model;
wherein the step of synthesizing the foot appearance data model and the foot muscle data model into a foot data model further comprises:
synthesizing the foot appearance data model, a foot bone data model and the foot muscle data model into the foot data model.

3. The method according to claim 1, further comprising:
combining the foot auxiliary equipment data model which is adjusted with the foot data model which is adjusted to form a wear data model;
performing a second dynamic analysis and a second static analysis on the wear data model;
determining whether the results of the second dynamic analysis and the results of the second static analysis are qualified; and
if the results of the second dynamic analysis and the results of the second static analysis are qualified, performing the step of printing the foot auxiliary equipment.

4. The method according to claim 1, further comprising:
lightening the foot auxiliary equipment data model.

5. The method according to claim 1, further comprising:
surface-finishing the foot auxiliary equipment data model.

6. The method according to claim 1, further comprising:
filling in the foot auxiliary equipment data model with different materials.

7. The method according to claim 1, further comprising:
printing the foot auxiliary equipment with different materials.
